(19) United States
(12) Patent Application Publication
(10) Pub. No.: US 2023/0072528 A1
(43) Pub. Date: Mar. 9, 2023

(54) METHODS FOR DISCONTINUING A TREATMENT WITH A TYROSINE KINASE INHIBITOR (TKI)

(71) Applicants: INSERM (Institut National de la Santé et de la Recherche Médicale), Paris (FR); ASSISTANCE PUBLIQUE-HÔPITAUX DE PARIS (APHP), Paris (FR); CENTRE HOSPITALIER UNIVERSITAIRE DE POITIERS, Poitiers (FR); UNIVERSITE DE POITIERS, Poitiers (FR); UNIVERSITÉ PARIS-EST CRÉTEIL VAL DE MARNE, Créteil (FR)

(72) Inventors: André HERBELIN, Poitiers (FR); Jean-Marc GOMBERT, Poitiers (FR); Emilie CAYSSIALS, Poitiers (FR); Alice BARBARIN, Archamps (FR); Lucie LEFEVRE, Archamps (FR); Lydia ROY, Creteil (FR)

(21) Appl. No.: 17/760,202

(22) PCT Filed: Feb. 4, 2021
(86) PCT No.: PCT/EP2021/052655

§ 371 Date: Aug. 5, 2022

(30) Foreign Application Priority Data
Feb. 5, 2020 (EP) 20305107.3

(51) Int. Cl. G01N 33/574; A61K 31/426; A61P 35/00

(52) U.S. Cl. CPC G01N 33/574; A61K 31/426; A61P 35/00; G01N 2800/7028

(57) ABSTRACT

The present invention relates to a method for discontinuing a treatment with a TKI by determining the number and/or frequency of innate CD8(+) T-cells in subject and concluding that the treatment with a TKI should be discontinued when the number and/or frequency of innate CD8 T-cells is higher than the predetermined reference value. Inventors evaluated whether innate CD8(+) T-cells are an early target of CML therapy success. Among peripheral blood effector CD8(+) T-cells, inventors shown that both number nd/or frequency and functional signatures of innate CD8(+) T-cells are enhanced as early as 3 months of therapy. Strikingly, they observe that patients with high innate CD8(+) T-cell number and/or frequency at 3 months and/or diagnosis achieve a DMR earlier than patients with low innate CD8(+) T-cell number. Furthermore, a higher number and/or frequency of high innate CD8(+) T-cell patients achieved a stable DMR for over 2 years. They have also observed that the success of TKI therapy cessation is associated with higher proportion of innate CD8 T-cells expressing perforin. Collectively, these findings highlight innate CD8(+) T-cells as a potential marker for both CML therapy success and successful long-term treatment-free remission (TFR), and thus therapy discontinuation eligibility.

### FIELD OF THE INVENTION

The invention is in the field of oncology. More particularly, the invention relates to a method for discontinuing a treatment with a TKI.

### BACKGROUND OF THE INVENTION

Chronic myeloid leukemia (CML) is a well-known model of immune system-sensitive disease as attested by old therapy successes: graft-versus-leukemia effect of allogenic-stem cell transplantation¹ or sustained remission after IFNα therapy in a subset of patients². Since the apparition of tyrosine kinase inhibitors (TKI), targeting specifically the BCR-ABL oncogene responsible of the disease, a high proportion of patients achieve complete cytogenetic response and major molecular response (MMR; BCRABL1/ABL1^{IS} ratio≤0.1%) leading to life expectancy close to age matched normal subjects³. After a couple of years of sustained deep molecular response (DMR; BCRABL1/ABL1^{IS} ratio≤0.01% or undetectable BCR-ABL1 with a least 10,000 copies of ABL1) under TKI treatment, some patients are offered an attempt to stop treatment, but in approximately 50% of cases, patients fail to maintain MMR and have to restart their TKI therapy⁴. This observation together with the evidence that TKI therapy fails to eliminate quiescent leukemic stem cells⁵ have renewed the interest for the immune control in CML.

Several studies showed that TKI have off-target effects on immune system. A particular interest was given to dasatinib, a second-generation TKI with broad Src kinase family inhibitory properties. In clinical practice, immunostimulatory effects have been observed, in particular the induction of large granular lymphocytes (LGL), consisting of cytotoxic T lymphocytes and natural killer (NK) cells^{6,7}. Several studies have also pointed the enhancement of NK-cell functions under dasatinib treatment⁸⁻¹².

Several studies report on immune markers that could be linked with outcomes of TKI cessation: Natural killer cells, CD86(+) plasmacytoid dendritic cells, γδ(+) T-cell−, CD4(+) CD25(+) CD127^{low} regulatory T cell-counts before TKI discontinuation. A recent study showed that CML patients in DMR under TKI therapy have normal immune effectors with a low expression of the inhibitory receptor PD-1, suggesting a restored functionality of immune effectors¹⁵. Most of the attention was given to NK cells as killer-cell immunoglobulin-like receptors (KIR) have been proposed as predictive markers for DMR^{16,17}, and several studies showed a link between NK-cell numbers and successful TKI cessation^{18,19}.

Thus, there is a need to better understand why in 50% of cases, patients maintain a sustained treatment-free remission (TFR) despite the persistence of leukemic cells.

### SUMMARY OF THE INVENTION

The invention refers to a method for diagnosing whether a subject will achieve a deep molecular response (DMR) with a tyrosine kinase inhibitor (TKI) comprising:

i) determining the number and/or frequency of innate CD8(+) T-cells in a biological sample obtained from the subject,

ii) comparing the number and/or frequency determined a step i) with a predetermined reference value and

iii) concluding that the subject will achieve a DMR when the number and/or frequency of innate CD8 T-cells is higher than the predetermined reference value or concluding that TKI treatment will not achieve a DMR when the number and/or frequency of innate CD8(+) T-cells is lower than the predetermined reference value.

The invention also refers to a method for discontinuing a treatment with a TKI comprising:

i) determining the number and/or frequency of innate CD8(+) T-cells in a biological sample obtained from the subject,

ii) comparing the number and/or frequency determined a step i) with a predetermined reference value and

iii) concluding that the treatment with a TKI should be discontinued when the number and/or frequency of innate CD8(+) T-cells is higher than the predetermined reference value or concluding that the treatment with a TKI should not be discontinued when the number and/or frequency of innate CD8(+) T-cells is lower than the predetermined reference value.

In particular, the invention is defined by claims.

### DETAILED DESCRIPTION OF THE INVENTION

By considering a cohort of patients receiving dasatinib and interferon-α in combination in which high rates of early and sustained DMR were achieved. Inventors evaluated whether innate CD8(+) T-cells are an early signal of CML therapy success. Among peripheral blood effector CD8(+) T-cells, inventors shown that both frequency and functional signatures of innate CD8(+) T-cells are enhanced as early as 3 months of therapy. Strikingly, they observe that patients with high innate CD8(+) T-cell frequency at 3 months and/or diagnosis achieve DMR earlier than patients with low innate CD8(+) T-cell frequency. Furthermore, patients with a higher proportion of CD8(+) T-cells have an increased probability of obtaining a stable DMR for over 2 years. They have also observe that the success of TKI therapy cessation is associated with higher proportion of innate CD8 T-cells expressing perforin.

Collectively, these findings highlight innate CD8(+) T-cells as a potential marker for both depth of response under TKI and therapy discontinuation eligibility (namely a successful long-term treatment-free remission (TFR)).

### Method for Diagnosing and/or Predicting Whether a Subject Suffering From a Cancer Will Achieve a DMR

Accordingly, in a first aspect, the invention relates to a method for diagnosing whether a subject will achieve deep molecular response (DMR) with a tyrosine kinase inhibitor (TKI) comprising:

i) determining the number and/or frequency of innate CD8(+) T-cells in a biological sample obtained from the subject,

ii) comparing the number and/or frequency determined a step i) with a predetermined reference value and

iii) concluding that the subject will achieve a DMR when the number and/or frequency of innate CD8(+) T-cells is higher than the predetermined reference value or concluding that TKI treatment will not achieve a DMR when the number and/or frequency of innate CD8(+) T-cells is lower than the predetermined reference value.

In a particular embodiment, the invention relates to a method for predicting whether a subject suffering from a cancer will achieve a deep molecular response (DMR) with a tyrosine kinase inhibitor (TKI) comprising:

i) determining the number and/or frequency of innate CD8(+) T-cells in a biological sample obtained from the subject,

ii) comparing the and/or frequency determined a step i) with a predetermined reference value and

iii) concluding that the subject will achieve a DMR with a TKI when the number and/or frequency of innate CD8(+) T-cells is higher than the predetermined reference value or concluding that the subject will not achieve a DMR with a TKI when the number and/or frequency of innate CD8(+) T-cells is lower than the predetermined reference value.

In another embodiment, the invention relates to a method for predicting a TKI treatment success-in a subject diagnosed to have a cancer comprising:

i) determining the number and/or frequency of innate CD8(+) T-cells in a biological sample obtained from the subject, ii) comparing the number and/or frequency determined a step i) with a predetermined reference value and

iii) concluding that TKI treatment will be success when the number and/or frequency of innate CD8(+) T-cells is higher than the predetermined reference value or concluding that TKI treatment will not be success when the number and/or frequency of innate CD8(+) T-cells is lower than the predetermined reference value.

As used herein term “diagnosing” refers to classifying a disease or a symptom, determining a severity of the disease, monitoring disease progression, forecasting an outcome of a disease and/or prospects of recovery.

As used herein, the terms “will achieve a deep molecular response” or “respond” refer to the response to a treatment of cancer in a subject. Typically such treatment induces, ameliorates or otherwise causes an improvement in the pathological symptoms, disease progression or physiological conditions associated with or resistance to succumbing to a disease such as a cancer. More particularly, such terms refers to an optimal response observed with a TKI treatment: major molecular response (MMR) is an important therapy goal because of its impact on the progression free survival while the achievement of a stable and deep molecular response in necessary to propose an attempt of TKI cessation.

Molecular monitoring in CML is performed by Real-time quantitative reverse transcription polymerase chain reaction (RQ-PCR) according to ELN recommendations. All results are expressed as the BCR-ABL1/ABL1 ratio in the international scale using a specific conversion factor calculated by a calibrator based on primary international reference material.

As used herein, the term “predicting” is intended herein the likelihood that a subject will respond or not to a tyrosine kinase inhibitor (TKI) and also the extent of the response. Predictive methods of the invention can be used clinically to make treatment decisions by choosing the most appropriate treatment modalities for any particular subject.

In particular, in the context of the invention, the term “respond” refers to the ability of a tyrosine kinase inhibitor (TKI) to an improvement of the pathological symptoms, thus, the subject presents a clinical improvement compared to the subject who does not receive the treatment. The said subject is considered as a “responder” to the treatment. The term “not respond” refers to a subject who does not present any clinical improvement to the treatment with a TKI. This subject is considered as a “non-responder” to the treatment. Accordingly, the subject as considered “non-responder” has a particular monitoring in the therapeutic regimen. In a particular embodiment, the response to a treatment is determined by Response evaluation criteria in solid tumors (RECIST) criteria. This criteria refers to a set of published rules that define when tumors in cancer subjects improve (“respond”), stay the same (“stabilize”), or worsen (“progress”) during treatment. In the context of the invention, when the subject is identified as responder, it means that said subject improves overall and progression-free survival (OS/PFS).

As used herein, the term “cancer” refers to a malignant growth or tumour resulting from an uncontrolled division of cells. The term “cancer” includes primary tumors and metastatic tumors.

In addition, the cancer may specifically be of the following histological type, though it is not limited to: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a particular embodiment, the cancer is a myeloproliferative neoplasm.

In a further embodiment, the cancer is chronic myeloid leukemia (CML). CML is a myeloproliferative disease that is driven by (9; 22) translocation in hematopoietic stem cells. It leads to the formation of the Philadelphia chromosome and the BCR-ABL1 fusion gene.

In a further embodiment, the cancer is ovarian cancer.

In a further embodiment, the cancer is breast cancer.

As used herein, the term “subject” denotes a mammal, such as a rodent, a feline, a canine, and a primate. Particularly, the subject according to the invention is a human. More particularly, the subject according to the invention has or is susceptible to have a cancer as described above.

In a particular embodiment, the subject has or is susceptible to have CML.

In a particular embodiment, the subject has or is susceptible to have an ovarian cancer.

In a particular embodiment, the subject has or is susceptible to have breast cancer.

In a further embodiment, the subject has or is susceptible to have myeloma.

In another embodiment, the subject has or is susceptible to have gastrointestinal stromal tumor (GIST).

In a particular embodiment, the subject is treated first-line with TKI for at least three months

In a particular embodiment, the subject is treated first-line with TKI for 3, 4, 5, 6, 7, 8, 9, 12, 13, 14, 15, 16, 17, 18, 19, 20, 21, 22, 23 or 24 months.

In a particular embodiment, the subject is treated first-line with TKI for three months.

In a particular embodiment, the subject is treated first-line with dasatinib for three months.

As used herein, the term “biological sample” refers to any sample obtained from a subject, such as a serum sample, a plasma sample, a urine sample, a blood sample, a lymph sample, a tissue sample, or a tumor sample. In a particular embodiment, biological sample for the determination of the number and/or the frequency of innate CD8(+) T-cells includes samples such as a blood sample, a lymph sample, or a biopsy. In a particular embodiment, the biological sample is a blood sample. In another embodiment, the biological sample is a plasma sample.

In a particular embodiment, the biological sample is peripheral blood mononuclear cells (PBMC). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis, which will preferentially lyse red blood cells. Such procedures are known to the experts in the art.

In a particular embodiment, the biological sample is a tissue sample such as tissue biopsy. More particularly, in a particular embodiment, the tissue sample is bone marrow. Typically, the bone marrow cells are isolated from the subjects suffering from a cancer. A skilled man knows different methods to isolate the bone marrow cells.

As used herein, the term “CD8(+) T-cells” refers to T cells expressing T-cell receptors (TCRs) that can recognize a specific antigen. More particularly, the term “CD8(+) T cells”, refers to T cells which carry the co-receptor CD8. CD8 is a transmembrane glycoprotein that serves as a co-receptor for the TCR. Like the TCR, CD8 binds to a major histocompatibility complex I (MHC I) molecule. “CD8(+) T-cells” differentiate into cytotoxic CD8(+) T-cells, and kill cancer cells and cells infected particularly by viruses by two ways. First way consists of releasing cytotoxins such as perforin and granzymes. The second way consist of inducing apoptosis by cell-surface interaction between the CD8(+) T-cells and the infected cell (FAS-L which is present on CD8(+) T-cells and FAS which is present on the target cell). Thus, CD8(+) T-cells are also called as cytotoxic T lymphocytes (CTL), T-killer cell, cytolytic T cells, CD8(+) T-cells or killer T-cells.

In a particular embodiment, the CD8(+) T-cells are innate CD8(+) T-cells. In the context of the invention, the term ‘innate CD8(+) T-cells” refers to CD8(+) T-cells responding to innate-like IL-12+IL-18 stimulation and co-expressing the transcription factor Eomesodermin (Eomes) and KIR/NKG2A membrane receptors with a memory/EMRA phenotype may represent a new, functionally distinct innate T cell subset in humans. In a particular embodiment, innate CD8(+) T-cells are defined by the expression of Eomesodermin, KIR/NKG2A membrane receptors. More particularly, in the context of the invention, the innate CD8(+) T-cells are defined by the expression of: KIR2D+KIR3DL1/KIR3DL2+NKG2A, referred to as pan-KIR/NKG2A. In a particular embodiment, KIR3DL2 is also called as CD158e/k. In a further embodiment, the NKG2A is also called as CD159a.

In a particular embodiment, the invention relates to a method for diagnosing whether a subject will achieve deep molecular response (DMR) with a tyrosine kinase inhibitor (TKI) comprising:

i) determining the number and/or frequency of innate CD8(+) T-cells in a biological sample obtained from the subject,

ii) determining the number and/or frequency of NK cells, Tregs, MSC and/or non-conventional T lymphocytes in a biological sample obtained from the subject,

iii) comparing the number and/or frequency of innate CD8(+) T-cells, determined a step i) with a predetermined reference value,

iv) comparing the number and/or frequency of NK cells, Tregs, MSC and/or non-conventional T lymphocytes determined a step ii) with a predetermined reference value and

v) concluding that the subject will achieve a DMR when the number and/or frequency of innate CD8(+) T-cells and/or of NK cells, Tregs, MSC and/or non-conventional T lymphocytes is higher than the predetermined reference value or concluding that the subject will not achieve a DMR when the number and/or frequency of innate CD8(+) T-cells, NK cells, Tregs, MSC and/or non-conventional T lymphocytes is lower than the predetermined reference value

In a particular embodiment, the invention relates to a method for predicting whether a subject suffering from a cancer will achieve a deep molecular response (DMR) with a tyrosine kinase inhibitor (TKI) comprising:

i) determining the number and/or frequency of innate CD8(+) T-cells in a biological sample obtained from the subject,

ii) determining the number and/or frequency of NK cells, Tregs, MSC and/or non-conventional T lymphocytes in a biological sample obtained from the subject,

iii) comparing the number and/or frequency of innate CD8(+) T-cells, determined at step i) with a predetermined reference value;

iv) comparing the number and/or frequency of NK cells, Tregs, MSC and/or non-conventional T lymphocytes determined a step ii) with a predetermined reference value and

v) concluding that the subject will achieve a DMR with a TKI when the number and/or frequency of innate CD8(+) T-cells and/or level of NK cells, Tregs, MSC and/or non-conventional T lymphocytes is higher than the predetermined reference value or concluding that the subject will not achieve a DMR with a TKI when the number and/or frequency of innate CD8(+) T-cells, NK cells, Tregs, MSC and/or non-conventional T lymphocytes is lower than the predetermined reference value.

In another embodiment, the invention relates to a method for predicting a TKI treatment success in a subject diagnosed to have a cancer comprising:

i) determining the number and/or frequency of innate CD8(+) T-cells in a biological sample obtained from the subject,

ii) determining the number and/or frequency of NK cells, Tregs, MSC and/or non-conventional T lymphocytes in a biological sample obtained from the subject,

iii) comparing the number and/or frequency of innate CD8(+) T-cells, determined at step i) with its predetermined reference value;

iv) comparing the number and/or frequency of NK cells, Tregs, MSC and/or non-conventional T lymphocytes determined a step ii) with its predetermined reference value and

v) concluding that TKI treatment will be success when the number and/or frequency of innate CD8(+) T-cells and/or the level of NK cells, Tregs, MSC and/or non-conventional T lymphocytes is higher than the predetermined reference value or concluding that TKI treatment will not be success when the number and/or frequency of innate CD8(+) T-cells, NK cells, Tregs, MSC and/or non-conventional T lymphocytes is lower than the predetermined reference value.

As used herein, the term “non-conventional T lymphocytes” refers to lymphocytes that express a γδ TCR, and commonly reside in an epithelial environment such as the skin, gastrointestinal tract, or genitourinary tract. Their role is to recognize infections and cancer cells, and regulate inflammatory responses that arise in these tissues. In a particular embodiment, the non-conventional T lymphocytes are invariant natural killer T (iNKT) cells. iNKT cells have phenotypic and functional capacities of a conventional T cell, as well as features of natural killer cells (cytolytic activity). iNKT cells recognize glycolipids when presented in the context of CD1d, and play a role in allergy, autoimmunity, and host defense against cancer and infections.

As used herein, “NK cells” refers to a sub-population of lymphocytes that is involved in innate or non-conventional immunity. NK cells can be identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self MHC/HLA antigens by the activation of specific cytolytic machinery, the ability to kill tumor cells or other diseased cells that express a ligand for NK activating receptors, and the ability to release protein molecules called cytokines that stimulate or inhibit the immune response (“NK cell activities”). Any subpopulation of NK cells will also be encompassed by the term NK cells. Within the context of this invention “active” NK cells designate biologically active NK cells, including NK cells having the capacity of lysing target cells or enhancing the immune function of other cells. For instance, an “active” NK cell can be able to kill cells that express a ligand for an activating NK receptor and/or fail to express MHC/HLA antigens recognized by a KIR on the NK cell.

As used herein, the term “Tregs” also known as suppressor T cells, refers to a subpopulation of T cells that modulate the immune system, maintain tolerance to self-antigens, and prevent autoimmune disease. Tregs express the biomarkers CD4, FOXP3, and CD25. Typically, Tregs are capable of suppressive activity (i.e. inhibiting proliferation of conventional T cells), either by cell-cell contact or by MLR suppression (Mixed Lymphocytes Reaction). These cells include different subpopulations including but not limited to, peripheral regulatory T cells, γδ regulatory T cells and invariant regulatory T cells.

As used herein, the term “MSC” called as “myeloid suppressive cells” refers to regulatory immune cells of myeloid lineage, which include, but are not limited to myeloid-derived suppressor cells (MDSCs), dendritic cells (DCs), and macrophages (Mreg). They are different from other myeloid cell types in which they possess immunosuppressive activities rather than immunostimulatory properties.

In a particular embodiment, the function innate CD8(+) T-cells is restored functionally. Indeed, inventors have observed that the innate CD8(+) T-cells release cytotoxins such as perforin, granzymes and IFNγ without presenting a TCR more precisely in response to a combination of the pro-inflammatory cytokines IL12+IL-18 (like NK cells).

Accordingly, in a particular embodiment, the method according to the invention further comprises a step of determining the level of perforin, granzymes and/or IFNγ among innate CD8(+) T-cells.

Accordingly, in a further embodiment, the invention relates to a method for diagnosing whether a subject will achieve deep molecular response (DMR) with a tyrosine kinase inhibitor (TKI) comprising:

i) determining the number and/or frequency of innate CD8(+) T-cells;

ii) determining the level of perforin, granzymes and/or IFNγ among innate CD8(+) T in a biological sample obtained from the subject,

iii) comparing the number and/or frequency of innate CD8(+) T-cells, determined a step i) with a predetermined reference value;

iv) comparing the level of perforin, granzymes and/or IFNγ among innate CD8(+) T-cells determined a step ii) with a predetermined reference value and

v) concluding that the subject will achieve a DMR when the number and/or frequency of innate CD8(+) T-cells and/or the level of perforin, granzymes and/or IFNγ among innate CD8(+) T-cells is higher than the predetermined reference value or concluding that the subject will not achieve a DMR when the number and/or frequency of innate CD8(+) T-cells and/or the level of perforin, granzymes and/or IFNγ among innate CD8(+) T-cells is lower than the predetermined reference value.

In a particular embodiment, the invention relates to a method for predicting whether a subject suffering from a cancer will achieve a deep molecular response (DMR) with a tyrosine kinase inhibitor (TKI) comprising:

i) determining the number and/or frequency of innate CD8(+) T-cells in a biological sample obtained from the subject,

ii) determining the level of perforin, granzymes and/or IFNγ among innate CD8(+) T-cells in a biological sample obtained from the subject,

iii) comparing the number and/or frequency of innate CD8(+) T-cells, determined at step i) with a predetermined reference value;

iv) comparing the level of perforin, granzymes and/or IFNγ among innate CD8(+) T-cells determined a step ii) with a predetermined reference value and

v) concluding that the subject will achieve a DMR with a TKI when the number and/or frequency of innate CD8(+) T-cells and/or level of perforin, granzymes and/or IFNγ among innate CD8(+) T-cells is higher than the predetermined reference value or concluding that the subject will not achieve a DMR with a TKI when the number and/or frequency of innate CD8(+) T-cells and/or level of perforin, granzymes and/or IFNγ among innate CD8(+) T-cells is lower than the predetermined reference value.

In another embodiment, the invention relates to a method for predicting a TKI treatment success in a subject diagnosed to have a cancer comprising:

i) determining the number and/or frequency of innate CD8(+) T-cells in a biological sample obtained from the subject,

ii) determining the level of perforin, granzymes and/or IFNγ among innate CD8(+) T-cells in a biological sample obtained from the subject,

iii) comparing the number and/or frequency of innate CD8(+) T-cells, determined at step i) with its predetermined reference value;

iv) comparing the level of perforin, granzymes and/or IFNγ among innate CD8(+) T-cells determined a step ii) with its predetermined reference value and

v) concluding that TKI treatment will be success when the number and/or frequency of innate CD8 T-cells and/or the level of perforin, granzymes and/or IFNγ among innate CD8(+) T-cells is higher than the predetermined reference value or concluding that TKI treatment will not be success when the number and/or frequency of innate CD8(+) T-cells and/or level of perforin, granzymes and/or IFNγ among innate CD8(+) T-cells is lower than the predetermined reference value.

The number and/or frequency of innate CD8(+) T-cells and/or other further immune cells is quantified by flow cytometry (immunophenotyping). This technique allows the analysis and sorting according to one or more parameters of the cells. All monoclonal antibodies (mAbs) used in this study are listed in Supplementary Table 2. Usually one or multiple secretion parameters can be analyzed simultaneously in combination with other measurable parameters of the cell, including, but not limited to, cell type, cell surface antigens, DNA content, etc. The data can be analyzed and cells can be sorted using any formula or combination of the measured parameters. Cell sorting and cell analysis methods are known in the art and are described in, for example, (The handbook of experimental immunology, Volumes 1 to 4, (D. N. Weir, editor) and flow cytometry and cell sorting (A. Radbruch, editor, Springer Verlag, 1992). Cells can also be analyzed using microscopy techniques including, for example, laser scanning microscopy, fluorescence microscopy; techniques such as these may also be used in combination with image analysis systems. Other methods for cell sorting include, for example, panning and separation using affinity techniques, including those techniques using solid supports such as plates, beads and columns. Some methods for cell sorting utilize magnetic separations, and some of these methods utilize magnetic beads. Different magnetic beads are available from a number of sources, including for example, Miltenyi Biotec GmbH (Germany).

In a further embodiment, the functionality of innate CD8(+) T-cells can be performed by three methods:

1) determination of the intracytoplasmic expression of IFN-γ by flow cytometry, after PBMC cultured for 24 h in the presence of the following cytokines cocktails: IL-12 and IL-18 or IL-12 and IL-33, intracellular transport inhibitor was added in the last 4 h of culture;

2) ADCC using anti-CD16 triggering, wells were precoated with 10 μg/mL Ultra-LEAF purified anti-human CD16 mAb or the corresponding isotype control provided by Biolegend and washed prior to addition of IL-15-presensitized PBMC. Cells were then incubated for 5 h.

3) Cytotoxic assays, IL-15-pretreated PBMC were incubated for 5 h with K562 target cells or autologous CD34(+) cells isolated at the time diagnosis of CML (in chronic phase) at a 1:5 ratio. For anti-CD16 triggering and cytotoxic assay, anti-CD107a mAb (or its corresponding IgG1 isotype control) and Golgistop (BD Biosciences) were added to cultures in the last 5 h.

In a particular embodiment, the immunometabolic state assessment of innate CD8(+) T-cells can be measured by two methods:

1) ex vivo flow cytometry using the combination of markers defined previously to identify innate CD8(+) T-cells with the following fluorescent dyes: 2-NBDG (2-(N-(7-Nitrobenz-2-oxa-1,3-diazol-4-yl)Amino)-2-Deoxyglucose) for the glucose uptake, MitoTrackers® for the mitochondrial mass and/or potential and BODIPY™ FL C16 or others lipid conjugated to fluorescent dye for lipid uptake

2) in vitro assessment of metabolic flux by measurement of media acidification and oxygen consumption using the Seahorse analyzer (Agilent Technologies) on live innate CD8 T-cells previously isolated with electronic cell sorting using the combination of markers defined previously to identify innate CD8(+) T-cells.

In a particular embodiment, the level, frequency, number or count of cells as described above (innate CD8(+) T-cells, NK cells, perforin, granzymes and/or IFNγ) can be measured by an immunoscore technic well known in the art, and described in Galon J, Fridman W H, Pages F: The adaptive immunologic microenvironment in colorectal cancer: a novel perspective. Cancer Res 2007, 67:1883-1886.

As used herein, the term “tyrosine kinase” refers to an enzyme that can transfer a phosphate group from ATP to a protein in a cell. Tyrosine kinases are responsible for catalyzing the transfer of a phosphoryl group from a nucleoside triphosphate donor, such as ATP, to an acceptor molecule. Tyrosine kinases catalyze the phosphorylation of tyrosine residues in proteins. The phosphorylation of tyrosine residues in turn causes a change in the function of the protein that they are contained in.

As used herein, the terms “tyrosine kinase inhibitor” or “TKI” refer to any compound, natural or synthetic, which results in a decreased phosphorylation of the tyrosine present on the intracellular domain of receptor tyrosine kinases (RTK) such as growth factor receptors.

Several generations of TKI are available. First generation Bcr-Abl tyrosine kinase inhibitors comprises imatinib. Second generation drugs are intended to have decreased resistance and intolerance than imatinib. Second generation drugs that are currently marketed are nilotinib and dasatinib, whereas bosutinib and ponatinib are third generation drugs. Said drugs are described in details below. Accordingly, the TKI is selected from the following group but not limited to: imatinib, nilotinib, dasatinib, ponatinib or bosutinib.

In a particular embodiment, the TKI is imatinib or 4-[(4-methylpiperazin-1-yl)methyl]-N-(4-methyl-3-{[4-(pyridin-3-yl)pyrimidin-2-yl]amino}phenyl)benzamide (marketed as GLIVEC® by Novartis and previously known as STI571) of formula (I):

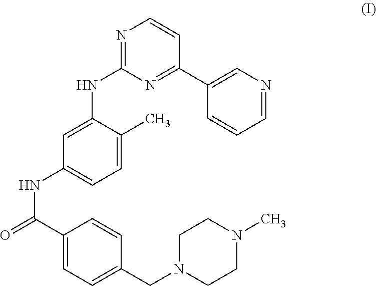 [SMILES: Cc1ccc(NC(=O)c2ccc(CN3CCN(C)CC3)cc2)cc1Nc1nccc(-c2cccnc2)n1]

In a particular embodiment, the TKI is nilotinib or 4-methyl-N-[3-(4-methyl-1H-imidazol-1-yl)-5-(trifluoromethyl)phenyl]-3-[(4-pyridin-3-ylpyrimidin-2-yl)amino]benzamide (marketed as TASIGNA® by Novartis and previously known as AMN107) of formula (II):

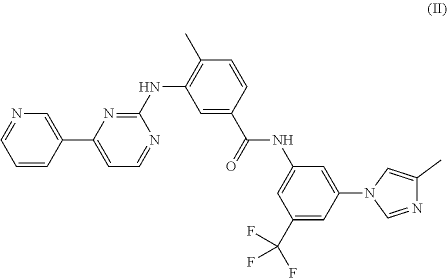 [SMILES: Cc1cn(-c2cc(NC(=O)c3ccc(C)c(Nc4nccc(-c5cccnc5)n4)c3)cc(C(F)(F)F)c2)cn1]

In a particular embodiment, the TKI is dasatinib or N-(2-chloro-6-methylphenyl)-2-[[6-[4-(2-hydroxyethyl)-1-piperazinyl]-2-methyl-4-pyrimidinyl]amino]-5-thiazole-carboxamide monohydrate (marketed as SPRYCEL® by BMS and previously known as BMS-354825) of formula (III):

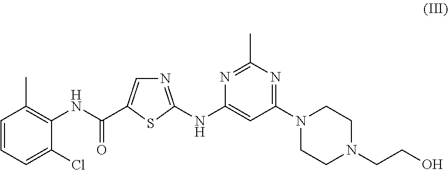 [SMILES: Cc1nc(Nc2ncc(C(=O)Nc3c(C)cccc3Cl)s2)cc(N2CCN(CCO)CC2)n1]

In a particular embodiment, the TKI is ponatinib or 3-(2-imidazo[1,2-b]pyridazin-3-ylethynyl)-4-methyl-N-[4-[(4-methylpiperazin-1-yl)methyl]-3-(trifluoromethyl)phenyl] benzamide (marketed as ICLUSIG® by and previously known as AP24534) of formula (IV):

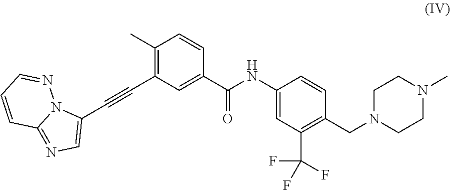 [SMILES: Cc1ccc(C(=O)Nc2ccc(CN3CCN(C)CC3)c(C(F)(F)F)c2)cc1C#Cc1cnc2cccnn12]

In a particular embodiment, the TKI is bosutinib or 4-[(2,4-dichloro-5-methoxyphenyl)amino]-6-methoxy-7-[3-(4-methylpiperazin-1-yl)propoxy]quinoline-3-carbonitrile (marketed as BOSULIF® by PFIZER and previously known as SM-606) of formula (V):

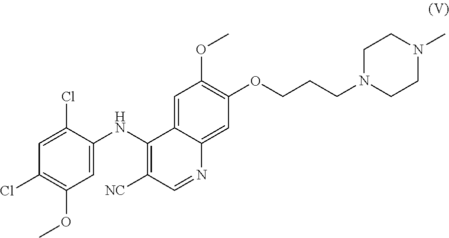 [SMILES: COc1cc(Nc2c(C#N)cnc3cc(OCCCN4CCN(C)CC4)c(OC)cc23)c(Cl)cc1Cl]

In a further embodiment, the method is suitable to diagnosis and/or predict whether a subject suffering from a cancer will achieve DMR with a TKI and a classical treatment, as a combined preparation.

In a further embodiment, the method is suitable to predict a TKI treatment success with a TKI and a classical treatment, as a combined preparation.

As used herein, the terms “combined preparation”, “combined therapy” or “therapy combination” refer to a treatment that uses more than one medication. The combined therapy may be dual therapy or bi-therapy. In the context of the invention, the cancer is treated with a combined treatment characterized by using a TKI and a classical treatment.

As used herein, the term “classical treatment” refers to treatments well known in the art and used to treat a cancer. In the context of the invention, the classical treatment refers to targeted therapy, radiation therapy, immunotherapy or chemotherapy.

In a particular embodiment, the method according to the invention is suitable to diagnosis and/or predict a DMR to a treatment with i) TKI and ii) an IFN_{α} used as a combined preparation.

In a particular embodiment, the method according to the invention is suitable to predict a TKI treatment success with i) TKI and ii) an IFN_{α} used as a combined preparation.

As used here, the tem “IFN” refers to interferons which belong to the large class of proteins known as cytokines, molecules used for communication between cells to trigger the protective defenses of the immune system that help eradicate pathogens.

IFN_{α} are mainly produced by plasmacytoid dendritic cells (pDCs). They are mainly involved in innate immunity against viral infection.

In a particular embodiment, the IFN_{α} is synthetically made for medication, the recombinant IFN_{α} is called as interferon alfacon-1. The pegylated types are pegylated interferon alfa-2a and pegylated interferon alfa-2b.

As used herein, the term “IFN_{α},” also known as HuIFN-alpha-Le, trade name Multiferon is a drug composed of natural interferon alpha (IFN-α). Interferon alfa (IFN-α) produced mainly by plasmacytoid dendritic cells (pDCs). They are mainly involved in innate immunity. The pegylated interferon alfa-2a, sold under the brand name Pegasys.

In a particular embodiment, the method according to the invention is suitable to diagnosis and/or predict a DMR to a treatment with i) TKI and ii) an Interferon alpha 2a (IFN-α 2a), used as a combined preparation.

In a particular embodiment, the method according to the invention is suitable to predict a TKI treatment success with i) TKI and ii) an Interferon alpha 2b (IFN-α 2b), used as a combined preparation.

As used herein, the term “IFN-α 2b” refers to a pegylated interferon alfa-2b, sold under the brand name Peglntron.

In a particular embodiment, the method according to the invention is suitable to diagnosis and/or predict a DMR to a treatment with i) TKI and ii) an inhibitor of BCR-ABL used as a combined preparation.

In a particular embodiment, the method according to the invention is suitable to predict a TKI treatment success with i) TKI and ii) an inhibitor of BCR-ABL used as a combined preparation.

As used herein, the term “BCR-ABL” refers to a constitutive tyrosine kinase. Its activity increases during proliferation and survival of myeloid progenitor cells. In a particular embodiment, the inhibitor of BCR-ABL is an allosteric kinase inhibitors. As used herein, the term allosteric inhibitor of BCR-ABL” refers to a compound that inhibits kinase activity by binding to a site remote from the active site of the kinase.

In a particular embodiment, the allosteric inhibitor of BCR-ABL is ABL001 also called as asciminib which is developed by Novartis. Asciminib and its derivatives are described in WO2013/171639 and has the following CAS number: 1492952-76-7.

In a particular embodiment, the method according to the invention is suitable to diagnosis and/or predict a DMR to a treatment with i) TKI and ii) an immune checkpoint inhibitor used as a combined preparation.

In a particular embodiment, the method according to the invention is suitable to predict a TKI treatment success with i) a TKI and ii) an immune checkpoint inhibitor used as a combined preparation.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8(+) T-cells from the naive to effector cell phenotype, however tumor-specific human CD8(+) T-cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8(+) T-cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4(+) T-cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophényl)-N′-hydroxy-4-{[2-(sulfamoylamino)-éthyl]amino}-1,2,5-oxadiazole-3 carboximidamide:

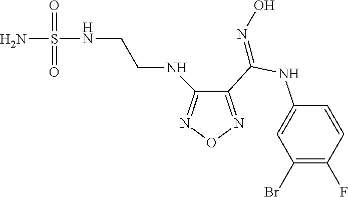 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

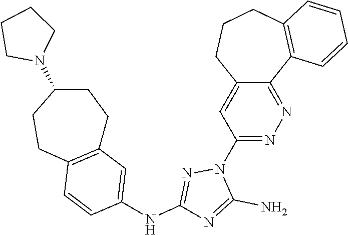 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with a high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

In another embodiment, the method according to the invention is suitable to diagnosis and/or predict a DMR to a treatment with i) a TKI and ii) a radiation therapy used as a combined preparation.

In a particular embodiment, the method according to the invention is suitable to predict a treatment success with i) a TKI and ii) a radiation therapy used as a combined preparation.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the method according to the invention is suitable to diagnosis and/or predict a DMR to a treatment with i) TKI and ii) a chemotherapy used as a combined preparation.

In a particular embodiment, the method according to the invention is suitable to predict a TKI treatment success with i) a TKI and ii) a chemotherapy used as a combined preparation.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

### Method for Discontinuing a Treatment With a TKI

Inventors have observed that a higher number of high innate CD8(+) T-cell patients achieved a stable DMR for over 2 years. Inventors have demonstrated that the success of TKI therapy cessation in a prospective treatment discontinuation cohort is associated with higher proportion of innate CD8 T-cells expressing perforin.

Collectively, these findings highlight innate CD8 T-cells as a potential marker for CML therapy discontinuation eligibility, and thus for a successful long-term treatment-free remission (TFR).

Accordingly, in a second aspect, the invention relates to a method for discontinuing a treatment with a TKI comprising:

i) determining the number and/or frequency of innate CD8(+) T-cells in a biological sample obtained from the subject,

ii) comparing the number and/or frequency determined a step i) with a predetermined reference value and

iii) concluding that the treatment with a TKI should be discontinued when the number and/or frequency of innate CD8(+) T-cells is higher than the predetermined reference value or concluding that the treatment with a TKI should not be discontinued when the number and/or frequency of innate CD8(+) T-cells is lower than the predetermined reference value.

As used herein, the terms “discontinuing” or “deprescribing” refer to the cession of a medication treatment. Typically, when the number and/or frequency of innate CD8(+) T-cells is higher than their predetermined reference value, the physician concludes to discontinue the treatment with a TKI. In the context of the invention, the term “discontinuing” means that the treatment with a TKI is a successful treatment and a successful TKI cessation and thus the subject is treatment free remission (TFR) from a cancer.

In a particular embodiment, the invention relates to a method for discontinuing a treatment with a TKI comprising:

i) determining the number and/or frequency of innate CD8(+) T-cells in a biological sample obtained from the subject,

ii) determining the number and/or frequency of NK cells, Tregs, MSC and/or non-conventional T lymphocytes in a biological sample obtained from the subject,

iii) comparing the number and/or frequency of innate CD8(+) T-cells, determined a step i) with a predetermined reference value,

iv) comparing the number and/or frequency of NK cells, Tregs, MSC and/or non-conventional T lymphocytes determined a step ii) with a predetermined reference value and

v) concluding that the treatment with a TKI should be discontinued when the number and/or frequency of innate CD8(+) T-cells, NK cells, Tregs, MSC and/or non-conventional T lymphocytes is higher than the predetermined reference value or concluding that the treatment with a TKI should not be discontinued when the number and/or frequency of innate CD8(+) T-cells, NK cells, Tregs, MSC and/or non-conventional T lymphocytes is lower than the predetermined reference value.

In a particular embodiment, the invention relates to a method for discontinuing a treatment with a TKI comprising:

i) determining the number and/or frequency of innate CD8(+) T-cells in a biological sample obtained from the subject,

ii) determining the level of perforin, granzymes and/or IFNγ among innate CD8(+) T-cells in a biological sample obtained from the subject,

iii) comparing the number and/or frequency of innate CD8(+) T-cells, determined a step i) with a predetermined reference value;

iv) comparing the level of perforin, granzymes and/or IFNγ among innate CD8(+) T-cells determined a step ii) with a predetermined reference value and

v) concluding that the treatment with a TKI should be discontinued when the number and/or frequency of innate CD8(+) T-cells and/or level of perforin, granzymes and/or IFNγ among innate CD8(+) T-cells is higher than the predetermined reference value or concluding that the treatment with a TKI should not be discontinued when the number and/or frequency of innate CD8(+) T-cells and/or level of perforin, granzymes and/or IFNγ among innate CD8(+) T-cells is lower than the predetermined reference value.

In a further embodiment, the method according to the invention is suitable to predict a treatment discontinuation when the subject is treated with i) a TKI and ii) a classical treatment, as a combined preparation.

In a particular embodiment, the method according to the invention is suitable to predict a treatment discontinuation when the subject is treated with i) TKI and ii) an IFN_{α} used as a combined preparation.

In a particular embodiment, the method according to the invention is suitable to predict a treatment discontinuation when the subject is treated with i) TKI and ii) an Interferon alpha 2a (IFN-α 2a), used as a combined preparation.

In a particular embodiment, the method according to the invention is suitable to predict a treatment discontinuation when the subject is treated with i) TKI and ii) an Interferon alpha 2b (IFN-α 2b), used as a combined preparation.

In a particular embodiment, the method according to the invention is suitable to predict a treatment discontinuation when the subject is treated with i) TKI and ii) an allosteric inhibitor of BCR-ABL used as a combined preparation.

In a particular embodiment, the method according to the invention is suitable to predict a treatment discontinuation when the subject is treated with i) TKI and ii) an immune checkpoint inhibitor used as a combined preparation.

In the context of the invention, when the method according to the invention concludes that the treatment with a TKI should be discontinued, it means that the subject will have a long survival time.

In the context of the invention, when the method according to the invention concludes that the treatment with a TKI should be discontinued, it means that the subject will have a good prognosis for a successful long-term treatment-free remission (TFR).

As used herein, the terms ‘number and/frequency” refers to the number of cells defined by certain marker molecules presented on the surface of these cells in relation to the number of cells of an entire definable population. In some embodiments, immune cells as described above are characterized herein by showing the cluster of differentiation and other markers such as CD8, KIR2D, KIR3DL1, KIR3DL2, NKG2A. Generally, for the methods disclosed herein, the number and/or frequency of a given immune population as described above is determined in relation to the total number/count of immune population in the biological sample.

As used herein, the term “predetermined reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of

a) providing a collection of biological samples from subject suffering from a cancer such as CML;

b) providing, for each biological sample provided at step a), information relating to the actual clinical outcome for the corresponding subject (i.e. the duration of the disease-free survival (DFS) and/or the overall survival (OS));

c) providing a serial of arbitrary quantification values;

d) quantifying the number of innate CD8(+) T-cells for each biological sample contained in the collection provided at step a);

e) classifying said biological sample in two groups for one specific arbitrary quantification value provided at step c), respectively: (i) a first group comprising biological sample that exhibit a quantification value for level that is lower than the said arbitrary quantification value contained in the said serial of quantification values; (ii) a second group comprising biological sample that exhibit a quantification value for said level that is higher than the said arbitrary quantification value contained in the said serial of quantification values; whereby two groups of biological sample are obtained for the said specific quantification value, wherein the biological sample of each group are separately enumerated;

f) calculating the statistical significance between (i) the quantification value obtained at step e) and (ii) the actual clinical outcome of the subjects from which biological sample contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant P-value obtained with a log-rank test, significance when P<0.05) has been calculated at step g).

For example the cell density has been assessed for 100 biological samples of 100 subjects. The 100 samples are ranked according to the number of cells. Sample 1 has the highest number and sample 100 has the lowest number. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan-Meier curves are prepared for each of the 99 groups of two subsets. Also for each of the 99 groups, the p value between both subsets was calculated (log-rank test). The predetermined reference value is then selected such as the discrimination based on the criterion of the minimum P-value is the strongest. In other terms, the cell density corresponding to the boundary between both subsets for which the P-value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P-value) are retained, so that a range of quantification values is provided. This range of quantification values includes a “cut-off” value as described above. For example, according to this specific embodiment of a “cut-off” value, the outcome can be determined by comparing the cell density with the range of values which are identified. In some embodiments, a cut-off value thus consists of a range of quantification values, e.g. centered on the quantification value for which the highest statistical significance value is found (e.g. generally the minimum P-value which is found).

### Method for Treating a Subject Identified as Responder With a TKI

In a third aspect, the invention relates to a method for treating a subject suffering from a cancer comprising the following steps: i) determining the number and/or frequency of innate CD8(+) T-cells in a biological sample obtained from the subject, ii) comparing the number and/or frequency determined a step i) with a predetermined reference value, iii) concluding that the subject will achieve a DMR with a TKI when the number and/or frequency of innate CD8(+) T-cells is higher than the predetermined reference value and iv) treating said subject with a TKI.

In a particular embodiment, the invention relates to a method a method for treating a subject suffering from a cancer comprising a step of administering a TKI to a subject identified as responder to a treatment with a TKI according to the method of the invention.

In a further embodiment, the invention relates to a method for treating a subject suffering from a cancer comprising the following steps:

i) determining the number and/or frequency of innate CD8(+) T-cells in a biological sample obtained from the subject,

ii) determining the number and/or frequency of NK cells, Tregs, MSC and/or non-conventional T lymphocytes in a biological sample obtained from the subject,

iii) comparing the number and/or frequency of innate CD8(+) T-cells, determined a step i) with a predetermined reference value,

iv) comparing the number and/or frequency of NK cells, Tregs, MSC and/or non-conventional T lymphocytes determined a step ii) with a predetermined reference value and

v) treating said subject with a TKI when the number and/or frequency of innate CD8(+) T-cells, NK cells, Tregs, MSC and/or non-conventional T lymphocytes is higher than the predetermined reference value or not treating said subject with a TKI when the number and/or frequency of innate CD8(+) T-cells, NK cells Tregs, MSC and/or non-conventional T lymphocytes is lower than the predetermined reference value.

In a further embodiment, the invention relates to a method for treating a subject suffering from a cancer comprising the following steps:

i) determining the number and/or frequency of innate CD8(+) T-cells in a biological sample obtained from the subject,

ii) determining the level of perforin, granzymes and/or IFNγ among innate CD8(+) T-cells in a biological sample obtained from the subject,

iii) comparing the number and/or frequency of innate CD8(+) T-cells, determined a step i) with a predetermined reference value;

iv) comparing the level of perforin, granzymes and/or IFNγ among innate CD8(+) T-cells determined a step ii) with a predetermined reference value and

v) treating said subject with a TKI when the number and/or frequency of innate CD8(+) T-cells and/or level of perforin, granzymes and/or IFNγ among innate CD8(+) T-cells is higher than the predetermined reference value or not treating said subject with a TKI when the number of innate CD8(+) T-cells and/or level of perforin, granzymes and/or IFNγ among innate CD8(+) T-cells is lower than the predetermined reference value.

In a further embodiment, the method according to the invention is suitable to treat a subject with i) a TKI and ii) a classical treatment, as a combined preparation when the subject is identified as having when:

the number and/or frequency of innate CD8(+) T-cells is higher than the predetermined reference value,

the number and/or frequency of innate CD8(+) T-cells NK cells, Tregs, MSC and/or non-conventional T lymphocytes is higher than the predetermined reference value;

the number and/or frequency of innate CD8(+) T-cells and/or level of perforin, granzymes and/or IFNγ among innate CD8(+) T-cells is higher than the predetermined reference value.

In a particular embodiment, the method according to the invention is suitable to treat a subject with i) TKI and ii) an IFN_{α} used as a combined preparation when:

the number and/or frequency of innate CD8(+) T-cells is higher than the predetermined reference value,

the number and/or frequency of innate CD8(+) T-cells, NK cells, Tregs, MSC and/or non-conventional T lymphocytes is higher than the predetermined reference value;

the number and/or frequency of innate CD8(+) T-cells and/or level of perforin, granzymes and/or IFNγ among innate CD8(+) T-cells is higher than the predetermined reference value.

In a particular embodiment, the method according to the invention is suitable to treat a subject with i) TKI and ii) an Interferon alpha 2a (IFN-α 2a), used as a combined preparation when:

i) the number and/or frequency of innate CD8(+) T-cells is higher than the predetermined reference value,

ii) the number and/or frequency of innate CD8(+) T-cells, NK cells, Tregs, MSC and/or non-conventional T lymphocytes is higher than the predetermined reference value; or

iii) the number and/or frequency of innate CD8(+) T-cells and/or level of perforin, granzymes and/or IFNγ among innate CD8(+) T-cells is higher than the predetermined reference value.

In a particular embodiment, the method according to the invention is suitable to treat a subject with i) TKI and ii) an Interferon alpha 2b (IFN-α 2b), used as a combined preparation when:

i) the number and/or frequency of innate CD8(+) T-cells is higher than the predetermined reference value,

ii) the number and/or frequency of innate CD8(+) T-cells, NK cells, Tregs, MSC and/or non-conventional T lymphocytes is higher than the predetermined reference value; or

iii) the number and/or frequency of innate CD8(+) T-cells and/or level of perforin, granzymes and/or IFNγ among innate CD8(+) T-cells is higher than the predetermined reference value.

In a particular embodiment, the method according to the invention is suitable to predict a treatment discontinuation when the subject is treated with i) TKI and ii) an allosteric inhibitor of BCR-ABL used as a combined preparation when:

i) the number and/or frequency of innate CD8(+) T-cells is higher than the predetermined reference value,

ii) the number and/or frequency of innate CD8(+) T-cells, NK cells, Tregs, MSC and/or non-conventional T lymphocytes is higher than the predetermined reference value; or

iii) the number and/or frequency of innate CD8(+) T-cells and/or level of perforin, granzymes and/or IFNγ among innate CD8(+) T-cells is higher than the predetermined reference value.

In a particular embodiment, the method according to the invention is suitable to predict a treatment discontinuation when the subject is treated with i) TKI and ii) an immune checkpoint inhibitor used as a combined preparation when:

i) the number and/or frequency of innate CD8(+) T-cells is higher than the predetermined reference value,

ii) the number and/or frequency of innate CD8(+) T-cells, NK cells and/or non-conventional T lymphocytes is higher than the predetermined reference value; or

iii) the number and/or frequency of innate CD8(+) T-cells and/or level of perforin, granzymes and/or IFNγ among innate CD8(+) T-cells is higher than the predetermined reference value.

As used herein, the terms “treating” or “treatment” refer to both prophylactic or preventive treatment as well as curative or disease modifying treatment, including treatment of subject at risk of contracting the disease (cancer) or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

### Pharmaceutical Composition

The TKI, IFN_{α}, IFN-α 2b, an allosteric inhibitor of BCR-ABL and/or an immune checkpoint inhibitor as described above may be combined with pharmaceutically acceptable excipients, and optionally sustained-release matrices, such as biodegradable polymers, to form pharmaceutical compositions.

Accordingly, in a fourth aspect, the invention relates to a pharmaceutical composition comprising TKI, IFN_{α}, IFN-α 2a, IFN-α 2b, an allosteric inhibitor of BCR-ABL and/or an immune checkpoint inhibitor and pharmaceutically acceptable excipients.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. Frequency and functional signatures of innate CD8(+) T-cells are impaired in patients at CML diagnosis. (A) Innate CD8(+) T-cell (Eomes+ panKIR/NKG2A+) frequency in CML patients at diagnosis (Diag, n=38) or in healthy donors (HD, n=21) among total CD8+TCR-αβ+ cells. Representative histograms of one HD and one CML patient are shown. (B) Eomes MFI (upper panel; Diag n=36; HD n=21) and CD49d MFI (lower panel; Diag n=32; HD n=20) in innate CD8 T-cells from CML patients at diagnosis or from HD. Representatives histograms are shown. Data are expressed as Box and whiskers (5 percentile, median and 95 percentile). Statistical analysis: unpaired two-tailed Mann-Whitney test.

FIG. 2. Innate CD8(+) T-cells are enhanced after 3 months of CML therapy. (A) Kinetics of innate CD8(+) T-cell frequencies in CML patients analyzed from diagnosis and up to 24 months of treatment (Diag: n=38; M3: n=34; M6: n=34; M12: n=33; M24: n=31). (B) Frequencies of innate CD8(+) T-cells at diagnosis (Diag, n=38) and after 3 months of CML therapy (M3, n=34). (C) Eomes MFI (upper panel; Diag n=36; M3 n=32) and CD49d MFI (lower panel; Diag n=32; M3 n=29) in innate CD8 T-cells at diagnosis (Diag) and after 3 months of CML therapy (M3). Data are expressed as curve of mean±SEM (panel A), as symbol and line (panel B) or as Box and whiskers (5 percentile, median and 95 percentile, panel C). Statistical analysis: unpaired two-tailed Mann-Whitney test (panel A) or paired two-tailed Wilcoxon test (panel B and C) were used.

FIG. 3. Early shift in CD8(+) T-cells after 3 months of therapy in CML patients. Frequencies of conventional-memory CD8(+) T-cells (Eomes⁺ panKIR/NKG2A⁻) (A) and naive CD8(+) T-cells (Eomes⁻ panKIR/NKG2A⁻ cells) (B) among total CD8⁺TCR-αβ⁺ cells in CML patients at diagnosis (Diag, n=38) or after 3 months of therapy (M3, n=34). Data are expressed as Box and whiskers (5 percentile, median and 95 percentile). Statistical analysis: a paired two-tailed Wilcoxon test was used.

FIG. 4. Achievement of a deep molecular response (DMR) at 12 months of CML therapy is associated with a high innate CD8(+) T-cell frequency at 3 months of treatment. (A) Frequencies of innate CD8(+) T-cells after 3 months of dasatinib treatment in patients having achieved DMR (M12-DMR, n=15) or not (M12-noDMR, n=12) after 12 months of therapy. (B) Eomes (upper panel, noDMR n=18; DMR n=14) and CD49d (lower panel, noDMR n=17; DMR n=12) MFI after 3 months of dasatinib treatment in patients having achieved DMR (M12-DMR) or not (M12-noDMR) after 12 months of therapy. (C) Kinetics of innate CD8(+) T-cell frequencies in CML patients having achieved DMR (M12-DMR, green line) or not (noDMR, blue line) after 12 months of therapy. Patients were analyzed from diagnosis and up to 24 months of treatment (Diag: noDMR n=22; DMR n=16; M3: noDMR n=19; DMR n=15; M6: noDMR n=20; DMR n=14; M12: noDMR n=18; DMR n=15; M24: noDMR n=17; DMR n=14). Data are expressed as Box and whiskers (5 percentile, median and 95 percentile, panel A and B) or as curve of mean±SEM (panel C). Statistical analysis: paired two-tailed Wilcoxon test (panel A) or unpaired two-tailed Mann-Whitney test (panel B and C). ns: not significative.

FIG. 5. High innate CD8(+) T-cell frequency in CML patients after 3 months of treatment is preferentially associated with an early and sustained DMR during CML therapy. (A) Cumulative incidence of the deep molecular response (DMR) over the first 24 months of therapy in patients with low (green line) or high (red line) innate CD8 T-cell frequency after 3 months of treatment. The number of subjects at risk are shown below the curve. (B) Frequencies of innate CD8(+) T-cells after 3 months of treatment in patients achieving a stable DMR over 2 years or not (<2-year DMR n=16; >2-year DMR n=18). (C) Conventional-memory (left) and naive (right) CD8(+) T-cell frequencies after 3 months of treatment in patients achieving a stable DMR over 2 years or not (<2-year DMR n=16; ≥2-year DMR n=18). Data are expressed with Kaplan-Meier curves (panel A) or as Box and whiskers (5 percentile, median and 95 percentile, panels B and C). Statistical analysis: Mantel-cox test was performed for cumulative incidence curve (panel A) and an unpaired two-tailed Mann-Whitney test for panels B and C. ns: not significative.

FIG. 6. High innate CD8(+) T-cell frequency in CML patients at diagnosis is preferentially associated with an early and sustained DMR. (A) Cumulative incidence of the deep molecular response (DMR) over the first 24 months of CML therapy in patients (the numbers of subjects at risk are shown below the curve) with low (green line) or high (red line) innate CD8(+) T-cell frequency at diagnosis. (B) Frequencies of innate CD8(+) T-cells at diagnosis in patients achieving a stable DMR over 2 years or not (<2-year DMR n=19; ≥2-year DMR n=18). (C) Conventional-memory (left) and naive (right) CD8(+) T-cell frequencies at diagnosis in patients achieving a stable DMR over 2 years or not (<2-year DMR n=19; ≥2-year DMR n=18). Data are expressed with Kaplan-Meier curves (panel A) or as Box and whiskers (5 percentile, median and 95 percentile, panels B and C). Statistical analysis: Mantel-cox test was performed for cumulative incidence curve (panel A) and an unpaired two-tailed Mann-Whitney test for panels B and C. ns: not significative.

FIG. 7: Successful TKI therapy cessation is associated with higher proportion of innate CD8 T-cells expressing perforin. Quantitative and functional analysis of innate CD8 LTs (Eomes+KIR/NKG2A+) in the prospective treatment discontinuation cohort. Proportion of innate CD8 LT expressing perforin was measured. Each symbol represents a healthy subject or a patient. Results are expressed as a percentage of total innate CD8 LT with representation of the median. Statistical analysis: Mann-Whitney test. HD (for a healthy donor): healthy donors (n=9), “relapse”: patients with molecular recurrence and resuming treatment during the first 6 months of stopping treatment (n=7), “no relapse”: patients in remission without treatment beyond 12 months of discontinuation (n=13).

### EXAMPLE 1

### Material & Methods

### 1. Patient and Healthy Donor Characteristics

The phase II DASA-PEGIFN clinical trial was registered with EudraCT number 2012-003389-42. Briefly, newly diagnosed Ph⁺ CP-CML patients were treated first-line with dasatinib at 100 mg/day. At 3 months, they were assigned to receive Peg-IFNα2b associated to dasatinib when platelets>100×109/L, neutrophils>1.5×109/L and lymphocytes<4.0×109/L counts were achieved. Venous blood was collected on heparin at several time points: diagnosis, 3, 6, 12 and 24 months after initiation of treatment Among the 61 patients receiving Dasatinib+Peg-IFNα2b therapy, we analysed samples from 40 patients. Two out of them were excluded for technical issues. See Table 1 for detailed patient's characteristics. Patients response to treatment were classified conforming to 2013 ELN criteria. Major molecular response was defined as a ratio of BCR-ABL1/ABL1≤0.1% on the international scale (IS). A ratio of BCR-ABL1/ABL1IS≤0.01% defined a molecular response of 4-log reduction or in our work a deep molecular response (DMR). All patients gave informed consent in accordance with the Declaration of Helsinki for participation in the study, which was approved by the scientific committee of the INSERM CIC-1402 (Poitiers, France) and Comité Protection Personnes Recherche Biomédicale Région Poitou Charentes (protocol number 12.10.31).

Frozen PBMC from healthy donors (HD) were obtained from the French Blood Institute (EFS, Lyon, France) (see Table 2).

### 2. PBMC Isolation and Cryopreservation

Peripheral blood mononuclear cells (PBMC) were isolated from blood samples by density gradient centrifugation (Histopaque®-1077, Sigma-Aldrich, St Louis, Mo., US), resuspended in 90% Fetal Bovine Serum (10270106, Gibco®, Thermo Fisher Scientific, Waltham, Mass., US) with 10% DMSO (D2650, Sigma-Aldrich), and cryopreserved at −80° C. or in liquid nitrogen until use.

### 3. Flow Cytometry

Phenotypic analysis of cells from HD and CML patients was performed by ex vivo flow cytometry. All monoclonal antibodies (mAbs) used in this study are listed in Supplementary Table 2. The expression of the different markers was assessed by staining PBMC with appropriate combinations of mAbs. panKIR/NKG2A referred to staining with the mix of the three following antibodies from Miltenyi Biotec (Bergisch Gladbach, Germany) KIR2D, KIR3DL1/KIR3DL2 (CD158e/k) and NKG2A (CD159a). Dead cells were excluded using the Live/Dead® Fixable NearIR Dead Cell Stain kit (L10119, Invitrogen™, Thermo Fisher Scientific). For intranuclear Eomes staining, cells were permeabilized with anti-human Foxp3 staining kit according to the manufacturer's protocol (73-5776-40, eBioscience™, Thermo Fisher Scientific). Flow data were acquired on a FACSVerse flow cytometer (Becton, Dickinson & Compagny, Franklin Lakes, N.J., US) with FACSuite™ software (Becton, Dickinson & Compagny) and analyzed using FlowJo™ v10 (Becton, Dickinson & Compagny). Results are expressed as either percent positive cells or as mean fluorescence intensity (MFI).

### 3. Statistical Analysis

Results are shown as box and whiskers with 5 percentile, median and 95 percentile unless indicated otherwise. All statistical data analyses were performed using GraphPad Prism v7.0 (GraphPad software, Inc). Wilcoxon and Mann-Whitney two-tailed test were used for paired and unpaired data analysis, respectively. A p value<0.05 was considered significant. Cumulative response rates were calculated using the cumulative incidence approach and Mantle-Cox method. The cut-off point for innate CD8(+) T-cells was optimized by receiver operating characteristics (ROC) curves and the Youden index using the logarithm of innate CD8(+) T-cell frequency at diagnosis and after 3 months of therapy. Patients were dichotomized according to their low or high percentage of innate CD8(+) T-cells. Data are presented as Kaplan-Meier curves.

TABLE 1
Dasa-PegIFN patient's characteristics

| | Patients eligible | | Selected patients for | |
|---|---|---|---|---|
| | to Peg-IFN after 3- | | immunological | |
| | month dasatinib | | analysis of this article | |
| | n = 61 | | n = 40 | |
| Median age, (range) | 45 | (20-65) | 48 | (20-65) |
| Pts <45 y, n (%) | 29 | (48) | 17 | (43) |
| Gender Male/Female, n (%) | 34 (56) 27 (44) | | 26 (65)/14 (35) | |
| Sokal, n (%) | | | | |
| Low | 30 | (51) | 22 | (55) |
| Int | 21 | (36) | 13 | (33) |
| High | 8 | (14) | 5 | (12) |

TABLE 2
Healthy donor's characteristics

| | HEALTHY DONORS | |
|---|---|---|
| | n = 21 | |
| Median age, (range) | 28 | (22-65) |
| Pts <45 y, n (%) | 16 | (76) |

| | |
|---|---|
| Gender Male/Female, n (%) | 7 (33)/14 (66) |

### Results

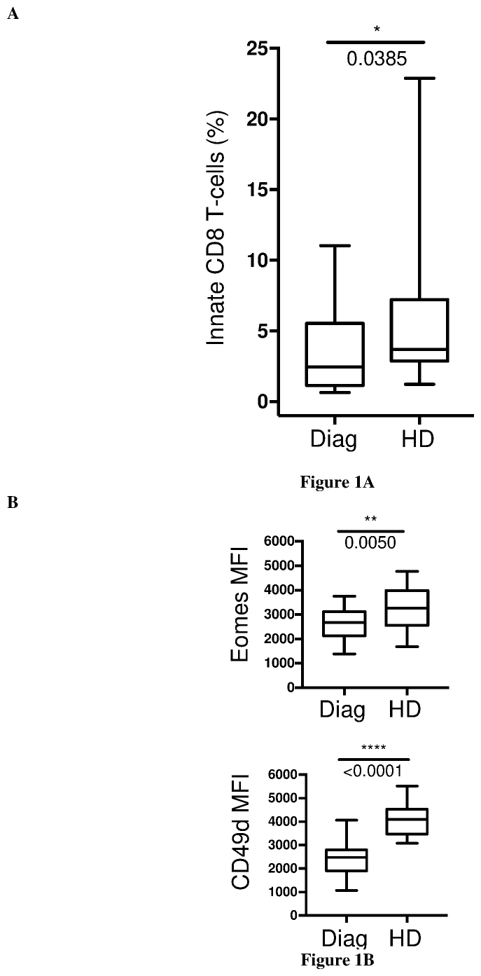

We analyzed innate CD8(+) T-cells (defined as Eomes+panKIR/NKG2A+ cells among TCR-αβ+ CD8+ cells) by flow cytometry from diagnosis and at several time points up to 24 months of DasaPegIFN therapy in 38 CML patients from the phase II clinical trial DASA-PEGIFN (see Methods, Table 1). In this group of patients, we found a lower frequency of innate CD8(+) T-cells at diagnosis as compared to the healthy donor (HD) group (FIG. 1A and data not shown). Moreover, innate CD8(+) T-cells showed a lower expression level of both the transcription factor Eomes and of the integrin CD49d (FIG. 1B and data not shown), two markers of the functional signature of these cells²¹. By contrast, frequencies of naive CD8(+) T-cells (defined as Eomes− panKIR/NKG2A−) and of conventional-memory CD8(+) T-cells (defined as Eomes+ panKIR/NKG2A−) in CML patients at diagnosis did not differ from those measured in HD (data not shown). These findings lead to definitively conclude that innate CD8(+) T-cells are specifically impaired in number and function at CML diagnosis.

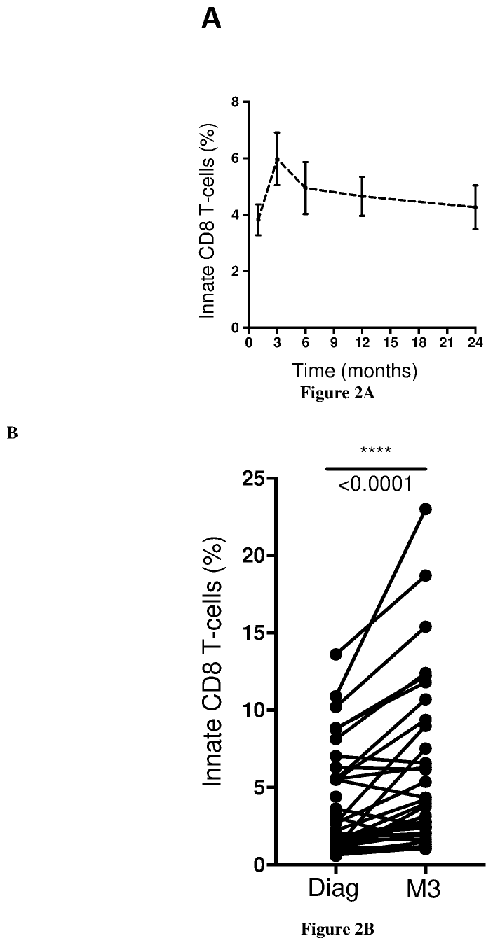

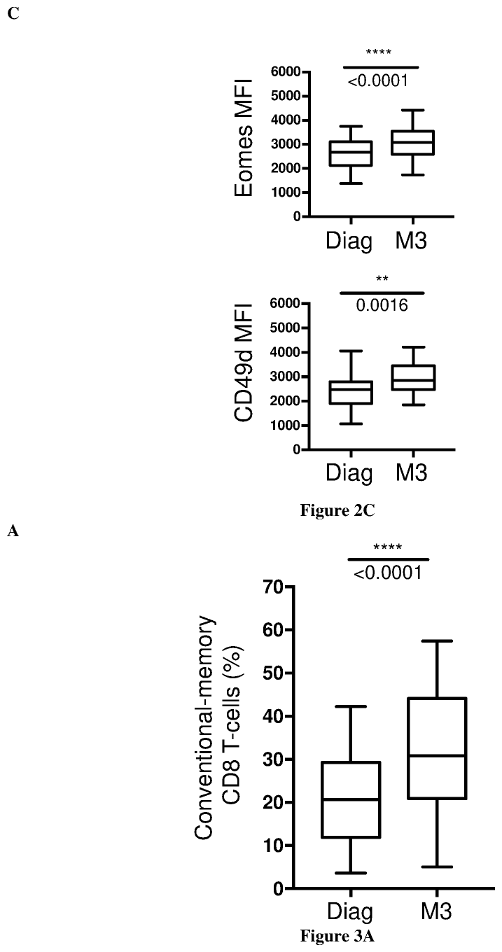

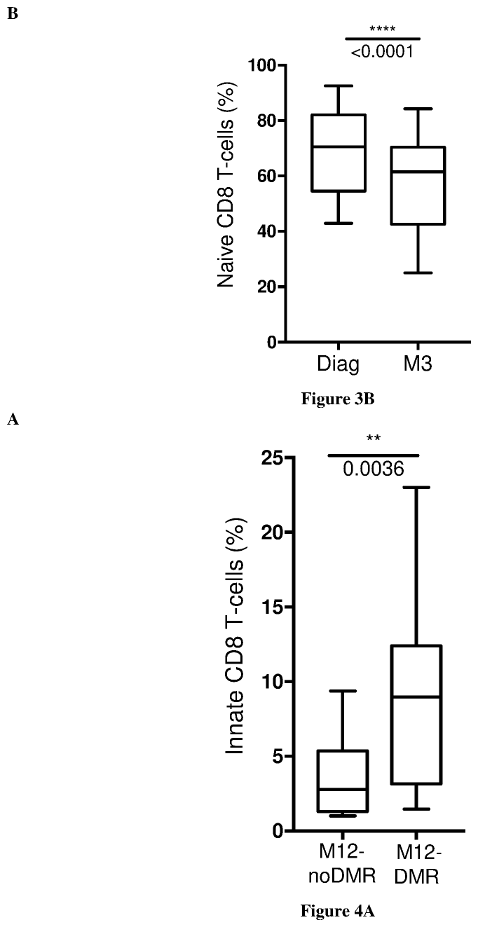

Knowing that innate CD8(+) T-cell deficiencies have been at least partially reversed in patients with TKI treatment over two years and having achieved a deep molecular response (DMR)²², we first analyzed whether normalization of this particular pool of cells could be reached in the first 24 months of therapy. The kinetics of innate CD8(+) T-cell frequencies from diagnosis to 24 months of therapy showed an increased frequency of innate CD8(+) T-cells as early as 3 months of therapy in the whole group (FIG. 2A). Focusing on this time point, we found a significant increase of the frequency of innate CD8(+) T-cells (5.98±5.43% vs 3.78±3.39%, mean±SD) (FIG. 2B) and of their Eomes and CD49d expression levels (FIG. 2C), as compared to diagnosis. Early effects of TKI therapy on the pool of CD8(+) T-cells were not restricted to innate CD8(+) T-cells, as conventional-memory CD8(+) T-cell frequency was concomitantly increased whereas naive CD8(+) T-cell frequency was decreased (FIG. 3A et B). Taken together, these findings strongly suggest an early shift within the pool of CD8(+) T-cells favoring an innate/memory phenotype in the first 3 months of therapy.

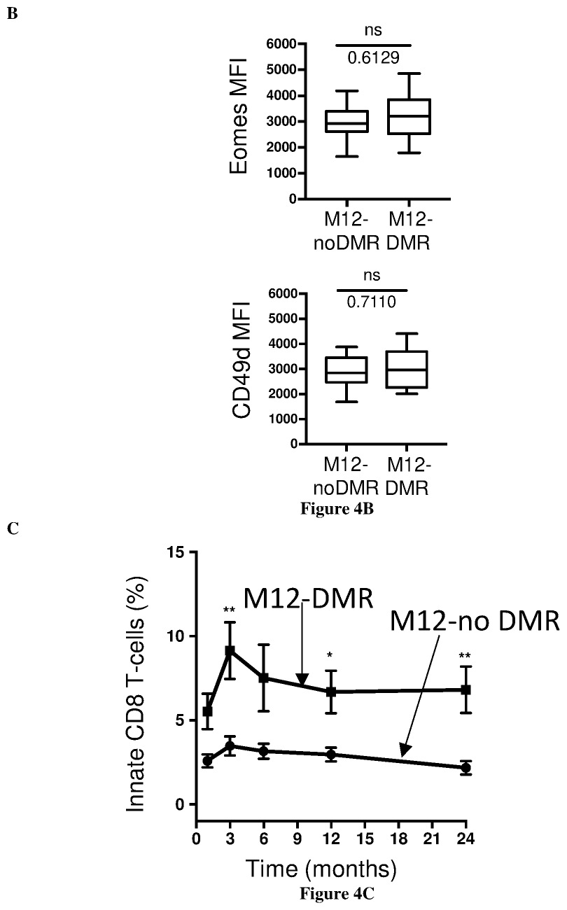

As innate CD8(+) T-cells were early impacted by CML therapy, we next searched for a link between their frequency and early DMR. To this end, we separated CML patients achieving DMR at 12 months (M12-DMR) or not (M12-noDMR). Remarkably, at 3 months after initiation of CML therapy, innate CD8(+) T-cell frequency in the M12-DMR group was nearly three-fold higher than in the M12-noDMR group (9.14±6.54% vs 3.48±2.43%, mean±SD) (FIG. 4A), even though it trended to be already higher than in the M12-noDMR group at CML diagnosis (FIG. 3A et B). At the 3-month time point, the higher frequency of innate CD8(+) T-cells in the M12-DMR group was not accompanied by a distinct functional status, as reflected by similar Eomes and CD49d expression levels in the two groups (FIG. 4B). This phenomenon was specific to innate CD8(+) T-cells, as conventional-memory CD8(+) T-cell frequency did not differ between the two groups (data not shown). Overall, the higher frequency of innate CD8 T-cells in the M12-DMR group was at the expense of their naive T-cell counterparts (data not shown), thereby suggesting a shift within the CD8(+) T-cell pool in favor of innate CD8(+) T-cells. Taking into account the whole kinetics, we evidenced that innate CD8(+) T-cell frequencies in M12-DMR patients were maintained at a higher level than in their M12-noDMR counterparts over the 24 months of therapy (FIG. 4C). Taken together, these results show for the first time that innate CD8(+) T-cells are associated with treatment success in patients with CML diagnosis.

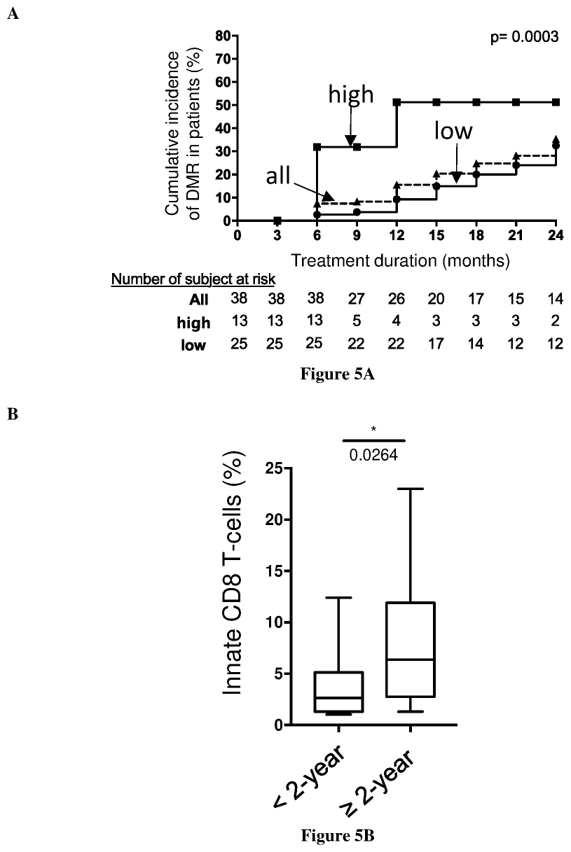

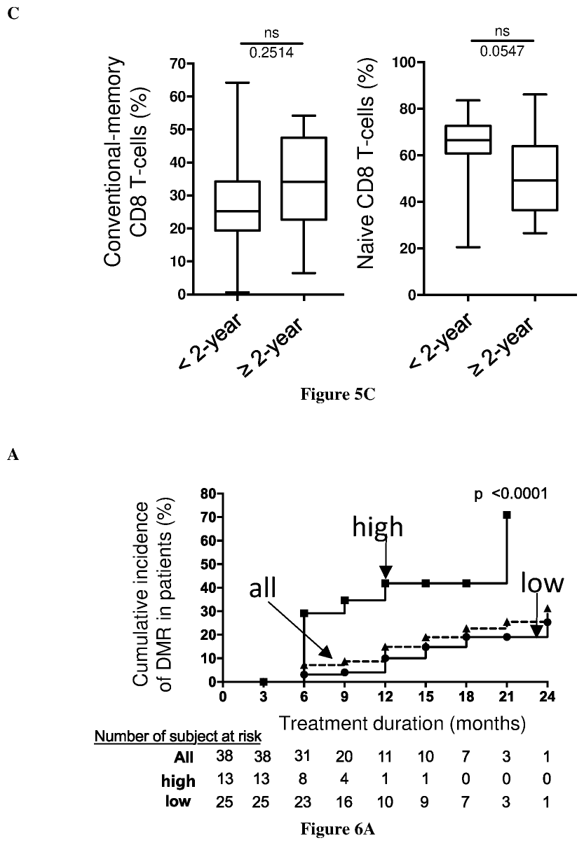

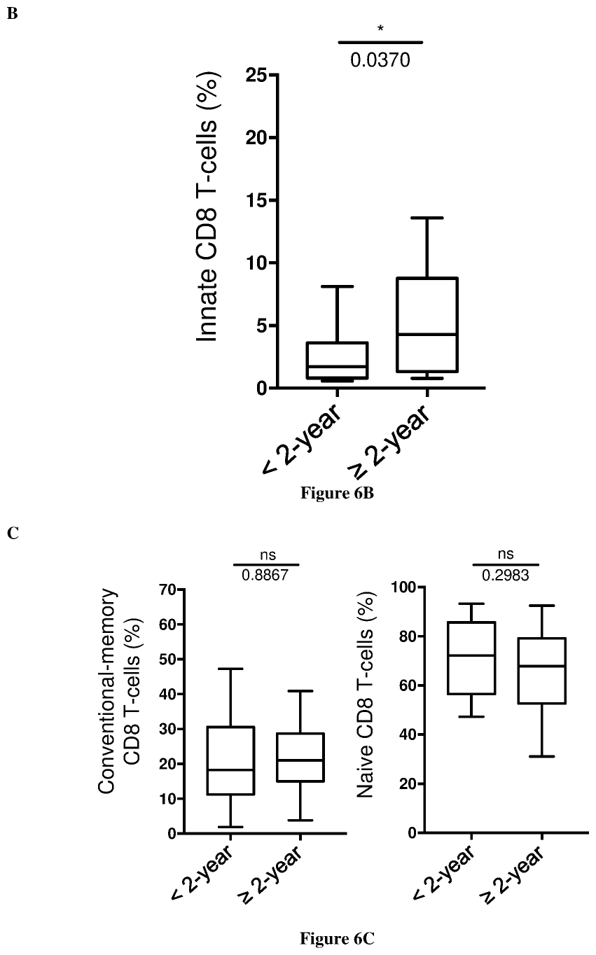

As innate CD8(+) T-cell frequency seemed to be an early target of CML therapy, we hypothesized that these cells are an indicator of early DMR achievement. Cumulative response rates of DMR occurred significantly earlier and at higher rates in innate CD8(+) T-cell high patients after 3-months therapy. Indeed, more than 50% of innate CD8(+) T-cell high patients achieved DMR at 12 months while less than 10% of innate CD8(+) T-cell low patients reached it (FIG. 5A). Interestingly, at 12-months the number of innate CD8(+) T-cell high patients reaching DMR remained stable up to 24-months of therapy whereas innate CD8(+) T-cell low patients continuously achieved DMR without reaching 50%. Moreover, taking into account innate CD8(+) T-cell frequency at CML diagnosis, cumulative response rates of DMR also occurred significantly earlier and at higher rates in innate CD8(+) T-cell high patients (FIG. 6A). These data lead to conclude that elevated innate CD8(+) T-cell frequency in peripheral blood both at diagnosis and at 3 months of therapy is an indicator of early DMR achievement.

Alongside DMR, its stability over time is another important criteria for CML patients. Indeed, patients achieving a stable DMR for two or more years are eligible to treatment discontinuation⁴. To test whether innate CD8(+) T-cells were associated with the stability of the treatment response, we separated patients having a stable DMR for over 2 years to patients without a stable DMR. Remarkably, after 3 months of therapy innate CD8(+) T-cell frequency was higher in patients having a stable DMR for over 2 years than in patients without a stable DMR (FIG. 5B). Once again, the same conclusion could be applied when considering the frequency of innate CD8(+) T-cells at CML diagnosis (FIG. 6B), thereby reinforcing the notion that the status of innate CD8(+) T-cells was closely associated with a stable DMR in CML patients. Finally, this effect was specific to innate CD8(+) T-cells, as conventional-memory and naive CD8(+) T-cell frequencies were similar between the two groups regardless of the time point (at diagnosis or after 3 months of therapy) (FIG. 5C and FIG. 6C). Collectively, our results show that innate CD8(+) T-cell numbers are associated with an early, deep and stable response to CML therapy.

### EXAMPLE 2

We prospectively investigated the quantitative and functional features of innate CD8 T-cells in patients who had stopped TKI according to the French group expert recommendations. The proportions of innate CD8 LTs (Eomes+MR/NKG2A+) expressing perforin were measured.

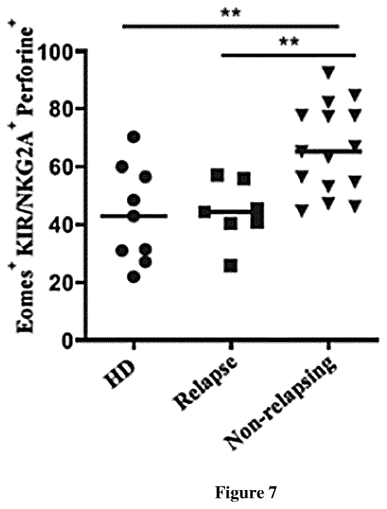

Our preliminary results (n=20) showed a significantly higher proportion of innate CD8 T-cells and of innate CD8 T-cells expressing perforin in TFR patients at M12 versus relapsing patients during the same period (FIG. 7). This result is in favour of the notion that the success of TKI therapy cessation is associated with higher proportion of innate CD8 T-cells expressing perforin.

Collectively, our findings highlight innate CD8 T-cells as a potential marker for both DMR achievement and successful long-term treatment-free remission (TFR).

### REFERENCES

Throughout this application, various references describe the state of the art to which this invention pertains. The disclosures of these references are hereby incorporated by reference into the present disclosure.

1 Chalandon Y, Passweg J R, Schmid C, Olavarria E, Dazzi F, Simula M P et al. Outcome of patients developing GVHD after DLI given to treat CML relapse: a study by the Chronic Leukemia Working Party of the EBMT. Bone Marrow Transplant 2010; 45: 558-564.

2 Bonifazi F, de Vivo A, Rosti G, Guilhot F, Guilhot J, Trabacchi E et al. Chronic myeloid leukemia and interferon-alpha: a study of complete cytogenetic responders. Blood 2001; 98: 3074-3081.

3 Bower H, Björkholm M, Dickman P W, Höglund M, Lambert P C, Andersson T M-L. Life Expectancy of Patients With Chronic Myeloid Leukemia Approaches the Life Expectancy of the General Population. J Clin Oncol Off J Am Soc Clin Oncol 2016; 34: 2851-2857.

4 Saussele S, Richter J, Guilhot J, Gruber F X, Hjorth-Hansen H, Almeida A et al. Discontinuation of tyrosine kinase inhibitor therapy in chronic myeloid leukaemia (EURO-SKI): a prespecified interim analysis of a prospective, multicentre, non-randomised, trial. Lancet Oncol 2018; 19: 747-757.

5 Corbin A S, Agarwal A, Loriaux M, Cortes J, Deininger M W, Druker B J. Human chronic myeloid leukemia stem cells are insensitive to imatinib despite inhibition of BCR-ABL activity. J Clin Invest 2011; 121: 396-409.

6 Mustjoki S, Ekblom M, Arstila T P, Dybedal I, Epling-Burnette P K, Guilhot F et al. Clonal expansion of T/NK-cells during tyrosine kinase inhibitor dasatinib therapy. Leukemia 2009; 23: 1398-1405.

7 Tanaka H, Nakashima S, Usuda M. Rapid and sustained increase of large granular lymphocytes and rare cytomegalovirus reactivation during dasatinib treatment in chronic myelogenous leukemia patients. Int J Hematol 2012; 96: 308-319.

8 Hassold N, Seystahl K, Kempf K, Urlaub D, Zekl M, Einsele H et al. Enhancement of natural killer cell effector functions against selected lymphoma and leukemia cell lines by dasatinib. Int J Cancer 2012; 131: E916-927.

9 Iriyama N, Takahashi H, Miura K, Uchino Y, Nakagawa M, Hata Y et al. Enhanced perforin expression associated with dasatinib therapy in natural killer cells. Leuk Res 2018; 68: 1-8.

10 Chang M-C, Cheng H-I, Hsu K, Hsu Y-N, Kao C-W, Chang Y-F et al. NKG2A Down-Regulation by Dasatinib Enhances Natural Killer Cytotoxicity and Accelerates Effective Treatment Responses in Patients With Chronic Myeloid Leukemia. Front Immunol 2018; 9: 3152.

11 Uchiyama T, Sato N, Narita M, Yamahira A, Iwabuchi M, Furukawa T et al. Direct effect of dasatinib on proliferation and cytotoxicity of natural killer cells in in vitro study. Hematol Oncol 2013; 31: 156-163.

12 Najima Y, Yoshida C, Iriyama N, Fujisawa S, Wakita H, Chiba S et al. Regulatory T cell inhibition by dasatinib is associated with natural killer cell differentiation and a favorable molecular response-The final results of the D-first study. Leuk Res 2018; 66: 66-72.

13 Sopper S, Mustjoki S, White D, Hughes T, Valent P, Burchert A et al. Reduced CD62L Expression on T Cells and Increased Soluble CD62L Levels Predict Molecular Response to Tyrosine Kinase Inhibitor Therapy in Early Chronic-Phase Chronic Myelogenous Leukemia. J Clin Oncol Off J Am Soc Clin Oncol 2017; 35: 175-184.

14 Schütz C, Inselmann S, Saussele S, Dietz C T, Mu Ller M C, Eigendorff E et al. Expression of the CTLA-4 ligand CD86 on plasmacytoid dendritic cells (pDC) predicts risk of disease recurrence after treatment discontinuation in CML. Leukemia 2017; 31: 829-836.

15 Hughes A, Clarson J, Tang C, Vidovic L, White D L, Hughes T P et al. CML patients with deep molecular responses to TKI have restored immune effectors and decreased PD-1 and immune suppressors. Blood 2017; 129: 1166-1176.

16 Kreutzman A, Jaatinen T, Greco D, Vakkila E, Richter J, Ekblom M et al. Killer-cell immunoglobulin-like receptor gene profile predicts good molecular response to dasatinib therapy in chronic myeloid leukemia. Exp Hematol 2012; 40: 906-913.e1.

17 Ureshino H, Shindo T, Kojima H, Kusunoki Y, Miyazaki Y, Tanaka H et al. Allelic Polymorphisms of KIRs and HLAs Predict Favorable Responses to Tyrosine Kinase Inhibitors in CML. Cancer Immunol Res 2018; 6: 745-754.

18 Ilander M, Olsson-Strömberg U, Schlums H, Guilhot J, Brück O, Lähteenmäki H et al. Increased proportion of mature NK cells is associated with successful imatinib discontinuation in chronic myeloid leukemia. Leukemia 2017; 31: 1108-1116.

19 Rea D, Henry G, Khaznadar Z, Etienne G, Guilhot F, Nicolini F et al. Natural killer-cell counts are associated with molecular relapse-free survival after imatinib discontinuation in chronic myeloid leukemia: the IMMUNOSTIM study. Haematologica 2017; 102: 1368-1377.

20 Jacomet F, Cayssials E, Basbous S, Levescot A, Piccirilli N, Desmier D et al. Evidence for eomesodermin-expressing innate-like CD8(+) KIR/NKG2A(+) T cells in human adults and cord blood samples. Eur J Immunol 2015; 45: 1926-1933.

21 Barbarin A, Cayssials E, Jacomet F, Nunez N G, Basbous S, Lefèvre L et al. Phenotype of NK-Like CD8(+) T Cells with Innate Features in Humans and Their Relevance in Cancer Diseases. Front Immunol 2017; 8. doi:10.3389/fimmu.2017.00316.

22 Jacomet F, Cayssials E, Barbarin A, Desmier D, Basbous S, Lefèvre L et al. The Hypothesis of the Human iNKT/Innate CD8(+) T-Cell Axis Applied to Cancer: Evidence for a Deficiency in Chronic Myeloid Leukemia. Front Immunol 2017; 7: 688.

1-9. (canceled)

10. A method for treating a subject suffering from a cancer comprising the following steps:
i) determining the number and/or frequency of innate CD8(+) T-cells in a biological sample obtained from the subject,
ii) determining that the number and/or frequency of innate CD8(+) T-cells in the biological sample is higher than a reference value, and
iii) treating the subject determined to have a higher number and/or frequency of innate CD8(+) T-cells with a TKI.

11. The method according to claim 10, wherein the subject is treated with i) a TKI and ii) IFN_{α}, IFN-α2a, IFN-α2b, an allosteric inhibitor of BCR-ABL or an immune checkpoint inhibitor as a combined preparation when:
the number and/or frequency of innate CD8(+) T-cells is higher than the reference value;
the number and/or frequency of innate CD8(+) T-cells is higher than the reference value and/or a level of NK cells is higher than a reference value and/or a level of non-conventional T lymphocytes is higher than a reference value; or
the number and/or frequency of innate CD8(+) T-cells is higher than the reference value and/or a level of perforin is higher than a reference value, and/or a level of granzymes is higher than a reference value and/or a level of IFNγ among innate CD8(+) T-cells is higher than a reference value.

12. The method according to claim 10, wherein the TKI is dasatinib.

13. The method according to claim 10, wherein the biological sample is a blood sample.

14. The method according to claim 10, wherein the innate CD8 T-cells are as KIR2D+KIR3DL1/KIR3DL2+NKG2A cells.

15. The method according to claim 10, wherein the subject is suffering or susceptible to suffer from a cancer.

16. The method according to claim 10, wherein the subject is first treated with dasatinib for three months.

17. The method according to claim 16, wherein the subject is subsequently treated with i) a TKI and ii) IFN_{α}, IFN-α 2a, IFN-α 2b, an allosteric inhibitor of BCR-ABL or an immune checkpoint inhibitor, as a combined preparation.
